(19) United States
(12) Patent Application Publication
(10) Pub. No.: US 2009/0043242 A1
(43) Pub. Date: Feb. 12, 2009

(54) INSTRUMENTS AND METHODS FOR IMPLANTING CORNEAL IMPLANT VIA EXTRA-AND INTRA-CAMERAL ROUTES

(75) Inventors: Eric A. Bene, Lynn, MA (US); Michael J. McGraw, Brighton, MA (US); Margaret Taylor, Groton, MA (US); Mark Bowen, Stow, MA (US); Ed Lee, Burlington, MA (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: Becton, Dickinson and Company, Franklin Lakes, NJ (US)

(21) Appl. No.: 11/835,070

(22) Filed: Aug. 7, 2007

(51) Int. Cl. A61M 27/00

(52) U.S. Cl. 604/8; 128/898; 606/108

(57) ABSTRACT

A method of implanting a transcorneal shunt into a cornea, the shunt having a head and a foot, each having a hole therein, the method including the operations of engaging an insertion tool with a foot hole of the shunt; making an entry incision in the cornea; inserting the shunt, while still engaged with the insertion tool, through the entry incision; making an implant incision in the cornea; inserting the head of the shunt through the implant incision to position and seat the shunt; and releasing the shunt from the insertion tool.

### BACKGROUND OF THE INVENTION

1. Field of the Invention

The present invention relates to devices and methods for use with ocular and non-ocular implants. More particularly, certain implementations of the present invention relate to insertion tools and methods for the controlled insertion of an ophthalmic shunt into an eye to relieve intraocular pressure via extra- and intra-corneal routes.

2. Description of the Related Art

Glaucoma, a condition caused by optic nerve cell degeneration, is the second leading cause of preventable blindness in the world today. In the human eye, aqueous humor is a transparent liquid that is constantly secreted by the ciliary body around the lens and flows into the region of the eye between the cornea and the lens, the anterior chamber. The trabecular meshwork provides the means by which the aqueous humor naturally drains from the anterior chamber. A major symptom of glaucoma is a high intraocular pressure, or “IOP,” which is caused by the trabecular meshwork failing to drain enough aqueous humor fluid from within the eye.

Conventional glaucoma therapy has been directed at protecting the optic nerve and preserving visual function by attempting to lower IOP using various methods, such as using drugs or surgery methods, including trabeculectomy and the use of implants. Trabeculectomy is a very invasive surgical procedure in which no device or implant is used. Typically, a surgical procedure is performed to puncture or reshape the trabecular meshwork by surgically creating a channel, thereby opening the sinus venosus.

Another surgical technique typically used involves the use of implants, such as stents or shunts, which are positioned within the eye and are typically relatively large. Such devices are implanted during any number of surgically invasive procedures, and serve to relieve internal eye pressure by permitting aqueous humor fluid to flow from the anterior chamber, through the sclera, and into a conjunctive bleb over the sclera. These procedures are very labor intensive for the surgeons and may be subject to failure due to scarring and cyst formations.

Another problem often related to the treatment of glaucoma with drugs relates to the challenge of delivering drugs to the eye. Current methods of delivering drugs to the eye are not as efficient or effective as desirable. Most drugs for the eye are applied in the form of eye drops, which have to penetrate through the cornea and into the eye. Drops are an inefficient way of delivering drugs; much of the drug never reaches the inside of the eye. Another treatment procedure includes injections. Drugs may be injected into the eye, but this is often traumatic and the eye typically needs to be injected on a regular basis.

One solution to the problems encountered with treatment of glaucoma using drops and injections involves the use of a transcorneal shunt, as disclosed herein. The transcorneal shunt is designed to be an effective means to reduce the intraocular pressure in the eye by shunting aqueous humor fluid from the anterior chamber of the eye. Surgical implantation of a transcorneal shunt is less invasive and quicker than other surgical options because the device is intended for implantation in the clear cornea. The transcorneal shunt drains aqueous humor fluid through the cornea to the tear film, rather than to the trabecular meshwork.

Additional details of ophthalmic shunts can be found, for example, in U.S. patent application Ser. No. 10/857,452, entitled “Ocular Implant and Methods for Making and Using Same,” filed Jun. 1, 2004 and published Jun. 2, 2005 under U.S. Publication No. 2005/0119737 A1, as well as International Patent Application No. PCT/US01/00350, entitled “Systems And Methods For Reducing Intraocular Pressure”, filed on Jan. 5, 2001 and published on Jul. 19, 2001 under the International Publication No. WO 01/50943. Details of ophthalmic shunts can also be found in U.S. Pat. No. 5,807,302, entitled “Treatment of Glaucoma,” filed Apr. 1, 1996 and issued Sep. 15, 1998. The entire contents of these applications and this patent are incorporated herein by reference.

### SUMMARY OF THE INVENTION

Accordingly, it is an aspect of the present invention to provide a transcorneal shunt and an insertion tool for use in shunt implantation. It is another aspect of the present invention to provide a method of implanting a transcorneal shunt.

The foregoing and/or other aspects of the present invention are achieved by providing an apparatus including: a transcorneal shunt with a head and a foot, each having a hole therein; and an insertion tool to insert the transcorneal shunt into a corneal incision. The insertion tool includes a stabilizer, and an engager protruding from the stabilizer and releasably engaging the shunt. When the stabilizer contacts one of the head and foot and surrounds the corresponding hole, the engager is inserted, at least partially, into the corresponding hole, and at least a portion of an inserted length of the engager includes an irregularity to enhance shunt engagement.

The foregoing and/or other aspects of the present invention are also achieved by providing an apparatus including: a hydrogel transcorneal shunt with a head and a foot, each having a hole therein; and an insertion tool to insert the transcorneal shunt into a corneal incision. The insertion tool includes a stabilizer, and an engager protruding from the stabilizer and releasably engaging the shunt. When the stabilizer contacts one of the head and foot and surrounds the corresponding hole, the engager is inserted, at least partially, into the corresponding hole. Additionally, the engager has at least a portion of an inserted portion thereof that is sized to be greater than a size, when the shunt is dehydrated, of the one of the head and foot holes the engager is inserted into, and less than the size, when the shunt is hydrated, of the one of the head and foot holes the engager is inserted into.

The foregoing and/or other aspects of the present invention are also achieved by providing an apparatus including: a transcorneal shunt with a head and a foot, each having a hole therein; and an insertion tool to insert the transcorneal shunt into a corneal incision. The insertion tool includes a stabilizer, and an engager protruding from the stabilizer and releasably engaging the shunt. The engager includes a hollow tube, and a plunger movably disposed within the hollow tube. When the stabilizer contacts one of the head and foot and surrounds the corresponding hole, the engager is inserted, at least partially, into the corresponding hole.

The foregoing and/or other aspects of the present invention are also achieved by providing an apparatus including: a hydrogel transcorneal shunt with a head and a foot, each having a hole therein; and an insertion tool to insert the transcorneal shunt into a corneal incision. The insertion tool includes: a handle to aid in manipulating the insertion tool; a stabilizer, including hypodermic tubing extending from the handle; and an engager protruding from the stabilizer and releasably engaging the shunt. The engager has a diameter greater than a diameter, when the shunt is dehydrated, of the of the one of the head and foot holes the engager is inserted into, and less than the diameter, when the shunt is hydrated, of the one of the head and foot holes the engager is inserted into.

The foregoing and/or other aspects of the present invention are also achieved by providing an apparatus including: a hydrogel transcorneal shunt with a head and a foot, each having a hole therein; and an insertion tool to insert the transcorneal shunt into a corneal incision. The insertion tool includes a stabilizer, and an engager protruding from the stabilizer and releasably engaging the shunt. The engager automatically releases the shunt subsequent the shunt's insertion into the corneal incision.

The foregoing and/or other aspects of the present invention are also achieved by providing a method of implanting a hydrogel transcorneal shunt into a cornea, the method including the operations: hydrating the shunt, the shunt having a head and a foot, each having a hole therein; inserting an engager of an insertion tool into one of the head and foot holes of the hydrated shunt; dehydrating the shunt subsequent to insertion of the engager; inserting the dehydrated shunt into a corneal incision; and re-hydrating the shunt to release the shunt from the insertion tool.

The foregoing and/or other aspects of the present invention are also achieved by providing a method of implanting a transcorneal shunt into a cornea, the method including the operations: inserting an engager of an insertion tool into one of a head and a foot hole of the shunt, and contacting a stabilizer of the insertion tool, from which the engager protrudes, to the one of the head and foot corresponding to the one of the head and foot holes the engager is inserted into, the engager including a hollow tube and a plunger that is movably disposed within the hollow tube; inserting a portion of the shunt through a corneal incision to position and seat the shunt; and releasing the shunt from the engager. Releasing the shunt from the engager includes one of extending the distal end of the plunger to a position outside of the hollow tube and retracting the plunger from the distal end of the hollow tube.

The foregoing and/or other aspects of the present invention are also achieved by providing a method of implanting a transcorneal shunt into a cornea, the shunt having a head and a foot, each having a hole therein, the method including the operations: engaging an insertion tool with a foot hole of the shunt; making an entry incision in the cornea; inserting the shunt, while still engaged with the insertion tool, through the entry incision; making an implant incision in the cornea; inserting the head of the shunt through the implant incision to position and seat the shunt; and releasing the shunt from the insertion tool.

The foregoing and/or other aspects of the present invention are also achieved by providing an apparatus including: a hydrogel transcorneal shunt with a head and a foot, each having a hole therein; and an insertion tool to insert the transcorneal shunt into a corneal incision. The insertion tool including a shaft portion, a stabilizing portion extending from the shaft portion, and an engaging portion extending from the stabilizing portion and releasably engaging the shunt. The engaging portion has at least a portion of an inserted portion thereof that is sized to be greater than a size of the foot hole when the shunt is dehydrated, and less than the size of the foot hole when the shunt is hydrated.

Additional and/or other aspects and advantages of the present invention will be set forth in part in the description that follows and, in part, will be apparent from the description, or may be learned by practice of the invention.

### BRIEF DESCRIPTION OF THE DRAWINGS

The above and/or other aspects and advantages of embodiments of the invention will become apparent and more readily appreciated from the following detailed description, taken in conjunction with the accompanying drawings, of which:

FIG. 1 illustrates a cross section of an ophthalmic shunt according to an embodiment of the present invention;

FIG. 2 illustrates a cross section of the ophthalmic shunt of FIG. 1 implanted in a cornea;

FIG. 3A illustrates an insertion tool according to an embodiment of the present invention;

FIG. 3B illustrates the ophthalmic shunt of FIG. 1 positioned on the insertion tool of FIG. 3A;

FIGS. 4A-4C illustrate cross sectional views of stabilizers of insertion tools according to embodiments of the present invention;

FIG. 5A illustrates an embodiment of the present invention;

FIG. 5B illustrates a perspective view of a portion of an engager of FIG. 5A;

FIG. 6A illustrates an embodiment of the present invention;

FIG. 6B illustrates a perspective view of a portion of an engager of FIG. 6A;

FIG. 7 illustrates an embodiment of the present invention in which a stabilizer and engager of an insertion tool are integrally formed as a unitary construction;

FIGS. 8A-8G illustrate variations of irregularities according to embodiments of the present invention;

FIG. 9 illustrates an embodiment of the present invention in which fluid is injected into an ophthalmic shunt via an insertion tool;

FIGS. 10A- 10C illustrate an apparatus and method according to an embodiment of the present invention, in which engagement and release of an ophthalmic shunt are mechanically directed;

FIGS. 11A-11C illustrate an apparatus and method according to another embodiment of the present invention, in which engagement and release of an ophthalmic shunt are mechanically directed;

FIG. 12A illustrates an embodiment of the present invention for use in intra-cameral implantation of a transcorneal shunt;

FIG. 12B illustrates a cross sectional detailed view of a circled region of FIG. 12A;

FIGS. 13-15 illustrate an apparatus and method according to an embodiment of the present invention for use in intra-cameral implantation of a transcorneal shunt;

FIG. 16 illustrates variations of insertion tools according to embodiments of the present invention; and

FIG. 17 illustrates a cross-sectional view of an incision-making device according to an embodiment of the present invention.

### DETAILED DESCRIPTION

Reference will now be made in detail to embodiments of the present invention, examples of which are illustrated in the accompanying drawings, wherein like reference numerals refer to the like elements throughout. The embodiments described explain the present invention by referring to the figures.

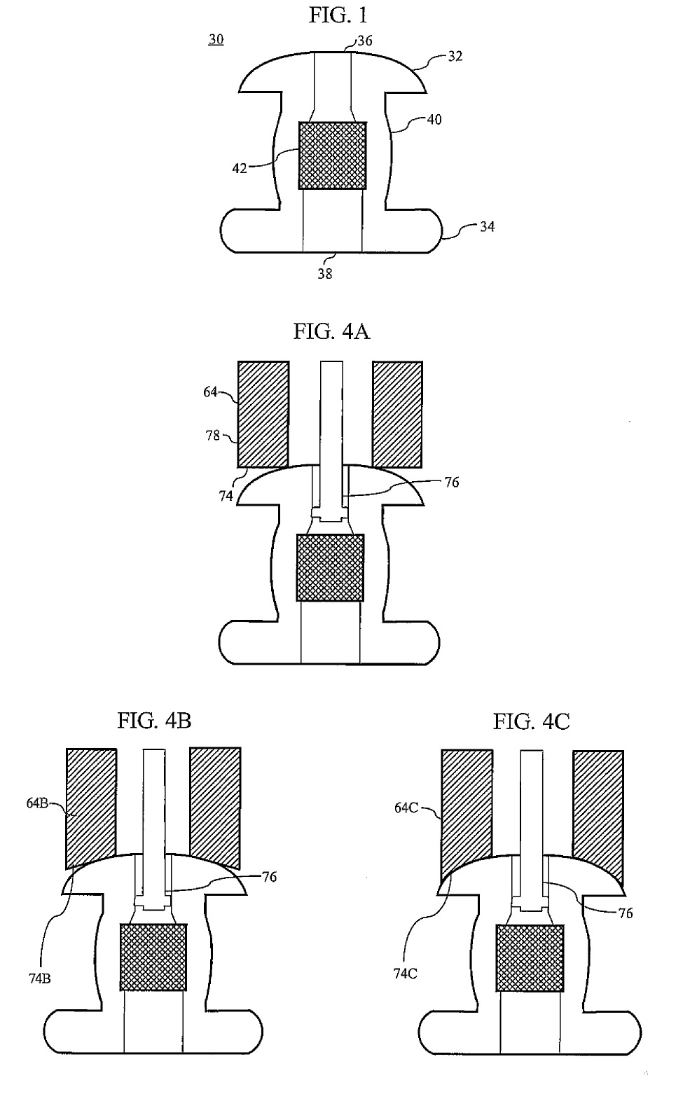

FIG. 1 illustrates a cross section of an ophthalmic shunt 30 according to an embodiment of the present invention, for example, a transcorneal shunt 30. The shunt 30 has a head 32 and a foot 34, each having the hole 36, 38 therein. A body 40 that forms a conduit extends between the head 32 and foot 34. According to one embodiment, the conduit 20 includes a filter 42 designed to restrict bacteria from infiltrating into the eye through the shunt and control a flow rate of aqueous humor from the anterior chamber of the eye to the outside surface of the cornea.

FIG. 2 illustrates a cross section of the ophthalmic shunt of FIG. 1 implanted in a cornea 50. As shown in FIG. 2, the transcorneal shunt 30 is inserted, or implanted, in the cornea 50 through a small incision. A surgeon selectively sizes the incision to allow the head 32 or the foot 34 to be manipulated through the incision, and yet prevent both the head 32 and the foot 34 from passing through once the shunt 30 is in place, thereby securing the shunt 30 in position. The head 32 anchors the shunt 30 on an outside surface 52 of the cornea 50, and the foot 34 anchors the shunt 30 on an inside surface 54 of the cornea 50. Sutures may be employed to aid in maintaining shunt implantation.

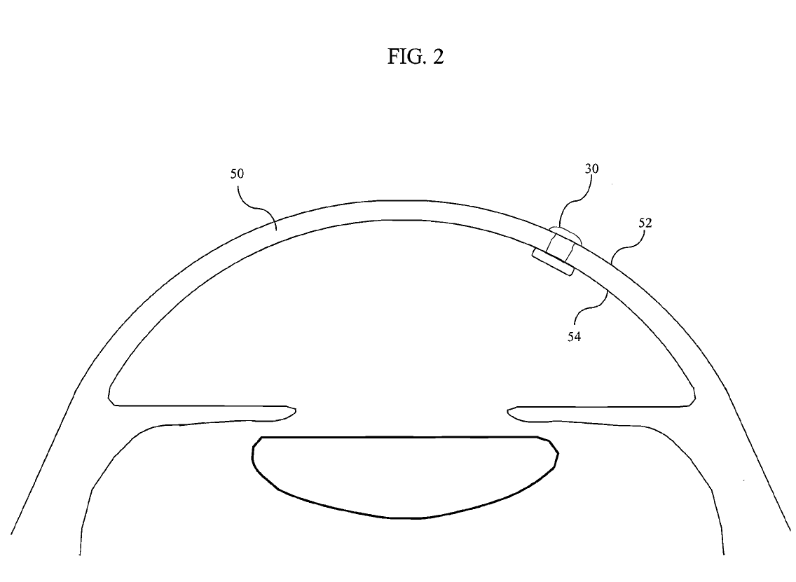

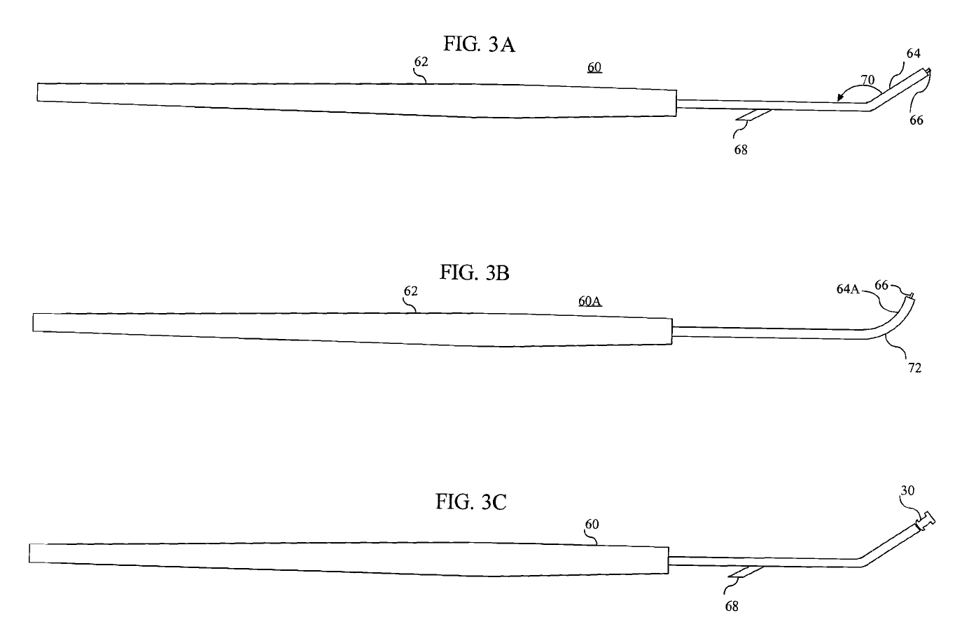

According to one embodiment, a surgeon, or other suitably trained person, uses an insertion tool to implant an ophthalmic shunt in a cornea. For brevity, such a person will hereinafter be referred to as a surgeon. One embodiment of such an insertion tool 60 is illustrated in FIGS. 3A and B. As shown in FIG. 3A, insertion tool 60 includes a handle 62 to aid manipulation of the insertion tool 60, a stabilizer 64 connected to the handle 62, and an engager 66 protruding from the stabilizer 64. Additionally, according to one embodiment, the stabilizer 64 includes hollow tubing. Further, according to one embodiment, the stabilizer 64 includes hypodermic tubing. Hollow and hypodermic tubing will be discussed in more detail later. Furthermore, FIG. 3A depicts an embodiment in which the insertion tool 60 includes a luer connection 68 to accommodate a syringe to inject fluid into the tubing.

Moreover, as depicted in FIG. 3A, a first end of the stabilizer 64, at which the engager 66 protrudes, has an angle 70 formed therein. Similarly, as depicted in FIG. 3B, a first end of the stabilizer 64A of insertion tool 60A, at which engager 66 protrudes, has a curve 72 formed therein. An advantage of the angle 70, or curve 72, is to make insertion of the shunt as comfortable and natural a motion as possible for the surgeon. FIG. 3C illustrates the ophthalmic shunt 30 of FIG. 1 positioned on the engager 66 of insertion tool 60 of FIG. 3A.

Focusing on the contact between the stabilizer and the shunt, FIGS. 4A-4C depict cross sectional views of stabilizers of insertion tools according to embodiments of the present invention in more detail. In addition to providing sturdiness for the contact between the insertion tool and the shunt, the stabilizer provides a hard stop, so that the engager, which protrudes from the stabilizer, cannot damage or displace the filter/flow restrictor of the shunt. FIGS. 4A-4C depict distal ends of stabilizers, at which engagers protrude. For clarity, a single embodiment of engager 76 is shown in FIGS. 4A-4C. But embodiments of the present invention are not limited to the engager 76 shown in FIGS. 4A-4C.

FIG. 4A illustrates stabilizer 64, as shown in FIG. 3A, in more detail. As shown in FIG. 4A, a distal end 74 of the stabilizer 64, at which the engager 76 protrudes, is approximately perpendicular to a side 78 of the stabilizer 64 immediately adjacent to the distal end 74 of the stabilizer 64.

FIG. 4B. illustrates a stabilizer 64B with a distal end 74B, at which engager 76 protrudes, that is recessed to accommodate a shape of the head 32. Similarly, FIG. 4C illustrates a stabilizer 64C with a distal end 74C, at which engager 76 protrudes, that is machined to a radius to conform to the shape of the head 32. In each of FIGS. 4A-4C, when the respective stabilizer contacts the head 32 and surrounds the corresponding hole (head hole 36), the engager 76 is inserted, at least partially, into the head hole 36.

Focusing now on the engagement between the engager and the shunt 30, FIG. 5A illustrates an embodiment of the present invention, in which engager 76, protruding from a stabilizer 80, has a portion that is inserted into head hole 36. And a portion of that inserted portion of the engager 76 includes an irregularity 82. According to one embodiment, as shown in FIG. 5A, the irregularity 82 includes a raised feature to releasably engage the shunt 30. Details of the engagement release between the shunt 30 and the irregularity will be discussed in more detail later.

Thus, FIG. 5A shows an apparatus having the following: a transcorneal shunt 30 with a head 32 and a foot 34, each having a hole 36, 38 therein; and an insertion tool 98 to insert the transcorneal shunt 30 into a corneal incision. The insertion tool 98 includes a stabilizer 80, and an engager 76 protruding from the stabilizer 80 and releasably engaging the shunt 30. Additionally, FIG. 5A shows that when the stabilizer 80 contacts the head 32 and surrounds the corresponding hole (head hole 36), the engager 76 is inserted, at least partially, into the head hole 36, and at least a portion of an inserted length of the engager 76 comprises an irregularity 82 to enhance shunt engagement.

FIG. 5B illustrates a perspective view of a portion of the engager 76 of FIG. 5A, and shows the raised feature irregularity.

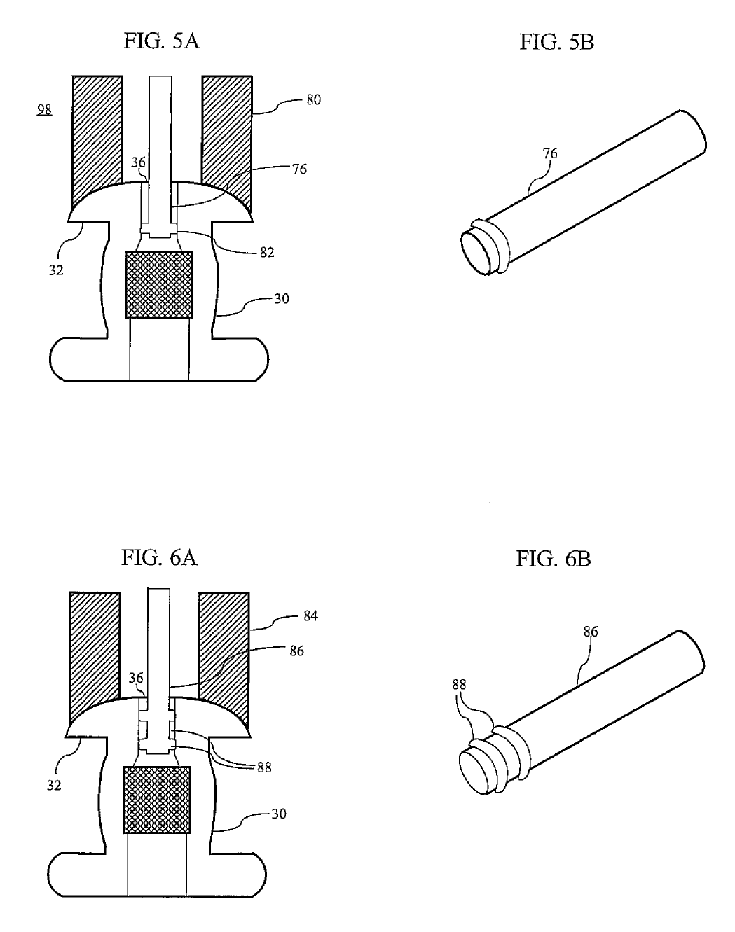

According to one embodiment, the engager includes a plurality of irregularities to enhance shunt engagement. FIG. 6A illustrates an embodiment of the present invention, in which engager 86, protruding from a stabilizer 84, has a portion that is inserted into head hole 36. And a portion of that inserted portion of the engager 86 includes a plurality of irregularities 88. FIG. 6C illustrates a perspective view of a portion of engager 86 of FIG. 6A, showing the plurality of irregularities 88. An additional advantage of the two irregularities 88 illustrated in this embodiment is to increase the stability of the shunt 30 on the insertion tool.

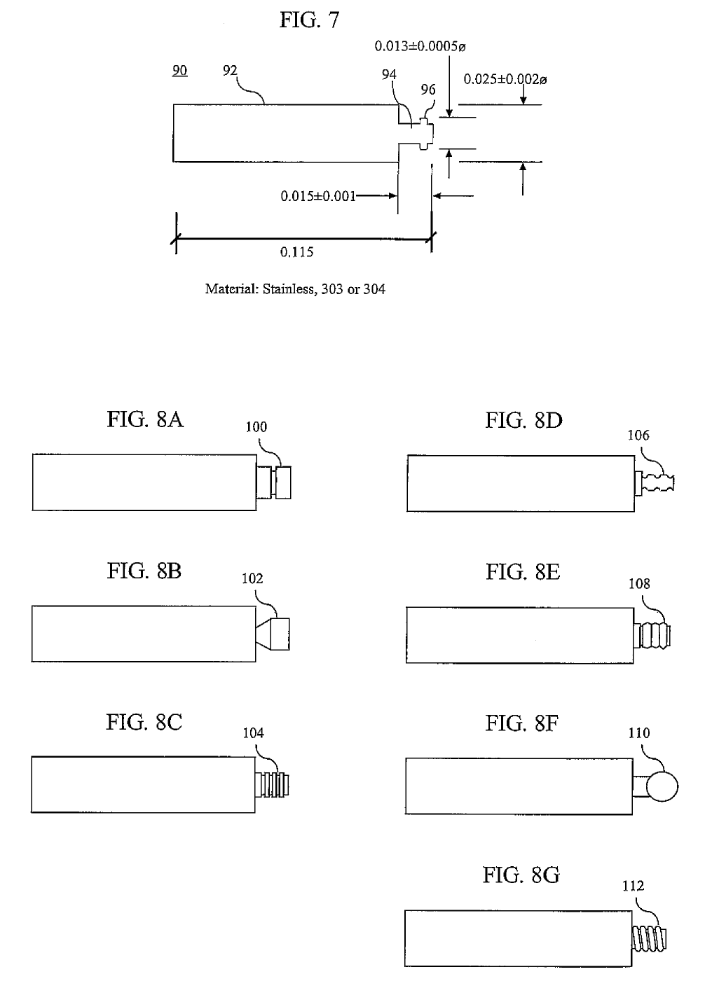

According to one embodiment, the stabilizer and the engager of the insertion tool are integrally formed as a unitary construction. FIG. 7 illustrates such an embodiment, in which stabilizer 92 and engager 94 of insertion tool 90 are integrally formed as a unitary construction. FIG. 7 also shows dimensions (in inches) of various portions of the insertion tool 90. For example, a length of the insertion tool 90 is shown as 0.115 in., the engager 94 extends 0.015 in. from the stabilizer 92, the irregularity 96 has a diameter of 0.013 in., and the stabilizer 92 has a diameter of 0.025 in.

As an example of an embodiment in which the stabilizer and the engager are integrally formed as a unitary construction, the stabilizer and engager could be milled from solid bar stock of stainless steel. Such an embodiment could then be used by a surgeon in concert with, for example, forceps, as the insertion tool, without the need for a handle, or a long stabilizer.

Up to this point, all the irregularities discussed have been raised features with respect to the engager. Interestingly, during experiments, researches unexpectedly discovered that nearly any irregularity on the engager enhances engagement with the shunt, even negative irregularities, for example, dimples or grooves.

FIGS. 8A-8G illustrate variations of irregularities in accordance with embodiments of the present invention, disposed on engagers of insertion tools in which the stabilizer and the engager of the insertion tool are integrally formed as a unitary construction. It will be understood, however, that these variations are not limited to embodiments in which the stabilizer and the engager of the insertion tool are integrally formed as a unitary construction. In FIG. 8A, the irregularity 100 includes a simple, square groove 100. In FIG. 8B, the irregularity 102 includes a tapered undercut 102. FIG. 8C shows the irregularity 104 as a plurality of square grooves 104. FIG. 8D illustrates the irregularity 106 as a plurality of rounded grooves 106. In FIG. 8E, the irregularity 108 includes a plurality of v-grooves 108. FIG. 8F shows the irregularity 110 as a ball-shape 1 10. And FIG. 8G illustrates the irregularity 112 as a helical thread 112.

Unexpectedly, it has been discovered that when employing irregularities on an engager, with respect to the stability of the shunt on the insertion tool, the importance of the closeness of the fit of the stabilizer to the head appears to diminish. In other words, when employing irregularities on an engager, with respect to the stability of the shunt on the insertion tool, the difference in performance among the embodiments shown, for example, in FIGS. 4A-4C, were not substantial. Therefore, a stabilizer such as that shown in FIG. 4A, which requires less machining than the stabilizer shown in FIG. 4C, can be used with similar effectiveness to the stabilizer shown in FIG. 4C, but at a reduced cost.

According to one embodiment, the ophthalmic shunt, for example, transcorneal shunt 30, is made of a hydrogel. Generally, hydrogels are soft, water-containing plastics (hydratable polymers). More specifically, hydrogels are networks of polymer chains that are water-insoluble, and are usually colloidal gels, in which water is the dispersion medium. Hydrogels are extremely absorbent and have a flexibility that is similar to that of natural tissue.

According to one embodiment, a hydrogel transcorneal shunt can be hydrated, dried, and re-hydrated. When the hydrogel shunt is hydrated, it becomes larger, and as it dries, it shrinks. According to one embodiment, the hydrogel shunt swells approximately 20% when it absorbs water, and returns to its original shape when dried. Employing this, one method of implanting a hydrogel transcorneal shunt into a cornea involves initially hydrating the shunt and loading the hydrated shunt onto an engager of an insertion tool, for example, insertion tool 98 shown in FIG. 5A. As shown in FIG. 5A, the engager 76 is inserted into head hole 36. When the shunt 30 dehydrates, the hydrogel shrinks, and creates an interference fit with the engager's 76 irregularity (in this case, raised feature) 82. Once dried (sufficiently dehydrated), the surgeon inserts the head 32 (intra-camerally) or foot 34 (extra-camerally) of the transcorneal shunt 30 into a corneal incision. Then, the subsequent re-hydration of the shunt 30 releases the shunt 30 from the insertion tool 98, and firmly seats the shunt 30 in the cornea.

Thus, according to one embodiment, a method of implanting a hydrogel transcorneal shunt into a cornea, includes the operations: hydrating the shunt; inserting an engager of an insertion tool into one of the head and foot holes of the hydrated shunt; dehydrating the shunt subsequent to insertion of the engager; inserting the dehydrated shunt into a corneal incision; and re-hydrating the shunt to release the shunt from the insertion tool.

To provide for the interference fit between the shunt and the engager and the release of the shunt from the insertion tool subsequent to the insertion into the corneal incision, the engager (and/or irregularity) and shunt are preferably sized in relation to one another. In other words, according to one embodiment, at least a portion of an inserted portion of the engager is sized to be greater than a size, when the shunt is dehydrated, of the one of the head and foot holes the engager is inserted into, and less than the size, when the shunt is hydrated, of the one of the head and foot holes the engager is inserted into. Stated another way, with respect to FIG. 5A, according to one embodiment, at least a portion (irregularity 82) of an inserted portion of the engager 76 is sized (diameter) to be greater than a size (diameter) of the head hole 36 when the shunt 30 is dehydrated, and less than the size (diameter) of the head hole 36, when the shunt 30 is hydrated.

Thus, FIG. 5A shows an apparatus having the following: a hydrogel transcorneal shunt 30 with a head 32 and a foot 34, each having a hole 36, 38 therein; and an insertion tool 98 to insert the transcorneal shunt 30 into a corneal incision. The insertion tool 98 includes a stabilizer 80, and an engager 76 protruding from the stabilizer 80 and releasably engaging the shunt 30. In addition, when the stabilizer 80 contacts the head 32 and surrounds the corresponding hole (head hole 36), the engager 76 is inserted, at least partially, into the corresponding hole (head hole 36). And the engager 76 has at least a portion 82 of an inserted portion thereof that is sized to be greater than a size, when the shunt 30 is dehydrated, of the one of the head and foot holes 36, 38 the engager 76 is inserted into, and less than the size, when the shunt 30 is hydrated, of the one of the head and foot holes 36, 38 the engager 76 is inserted into.

Regarding re-hydration of a shunt subsequent to insertion into a corneal incision, one method employs the aqueous humor inside the eye, thereby automatically releasing the shunt from the insertion tool.

Thus, FIG. 5A shows an apparatus having the following: a hydrogel transcorneal shunt 30 with a head 32 and a foot 34, each having a hole 36, 38 therein; and an insertion tool 98 to insert the transcorneal shunt 30 into a corneal incision. The insertion tool 98 includes a stabilizer 80, and an engager 76 protruding from the stabilizer 80 and releasably engaging the shunt 30, the engager 76 automatically releasing the shunt 30 subsequent the shunt's 30 insertion into the corneal incision.

Further, an additional way to hydrate a hydrogel transcorneal shunt has been developed. According to one embodiment, in which the stabilizer includes hollow tubing, for example hypodermic tubing, the stabilizer also includes a luer connection to accommodate a syringe to inject fluid into the tubing. An example of such a luer connection can be seen, for example, in FIG, 3A. Such a luer connection may be disposed on a side of the insertion tool (as depicted in FIG. 3A), or may be disposed at the proximal (with respect to the surgeon) end of the insertion tool (as opposed to the distal end at which the engager is disposed). The luer connection allows the surgeon to inject water or saline into the shunt during the implantation procedure. It is believed that this may speed up the hydration process of the shunt, at least at the critical point of the interference fit with the engager.

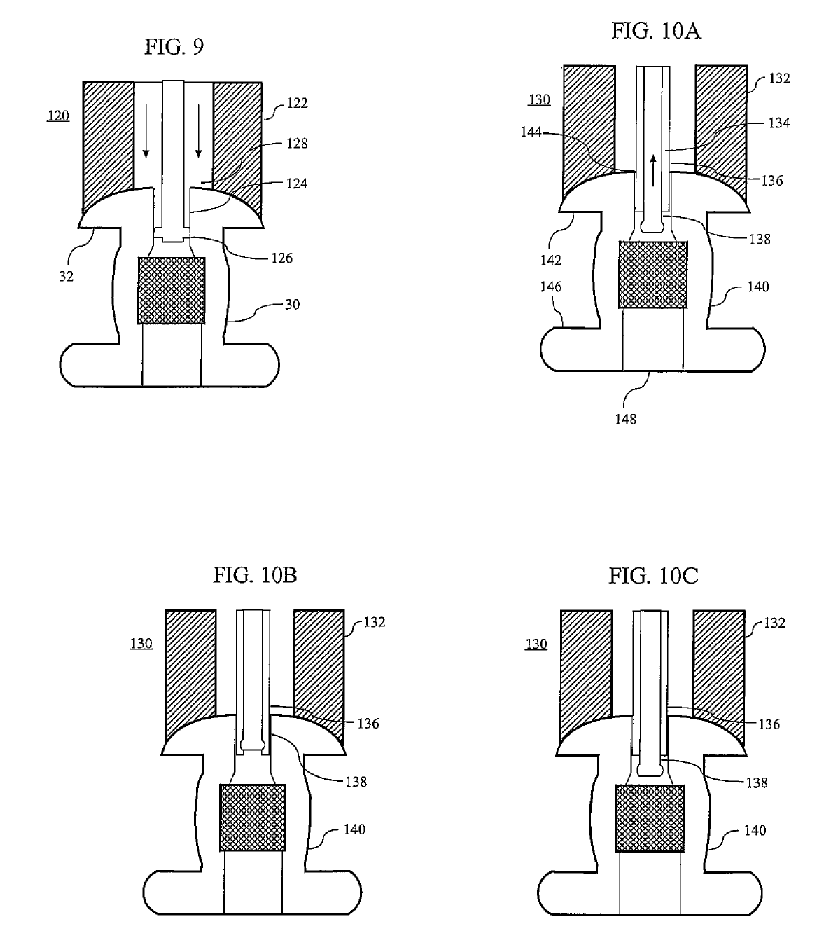

FIG. 9 illustrates an embodiment of the present invention in which fluid is injected into an ophthalmic shunt via an insertion tool. In FIG. 9, insertion tool 120 includes a stabilizer 122 and an engager 126 with an irregularity 126. Additionally, in this embodiment, though not shown for space considerations, the insertion tool 120 has a luer connection and a handle. Examples of similar luer connections and handles are illustrated, for example, in FIG. 3A. Further, in this embodiment, stabilizer 122 includes hypodermic tubing 122. FIG. 9 shows fluid 128 being injected into hydrogel transcorneal shunt 30 via hypodermic tubing 122.

Thus, FIG. 9 (in conjunction with portions of FIG. 3A) shows an apparatus having the following: a hydrogel transcorneal shunt 30 with a head 32 and a foot 34, each having a hole 36, 38 therein; and an insertion tool 120 to insert the transcorneal shunt 30 into a corneal incision. The insertion tool 120 includes a handle, a stabilizer 122, including hypodermic tubing 122, extending from the handle, and an engager 124 protruding from the stabilizer 122 and releasably engaging the shunt 30. The engager 124 has a diameter greater than a diameter, when the shunt 30 is dehydrated, of the of the one of the head and foot holes 36, 38 the engager 124 is inserted into, and less than the diameter, when the shunt 30 is hydrated, of the one of the head and foot holes 36, 38 the engager 124 is inserted into.

Examples of materials that can be used to manufacture a stabilizer and/or an engager in accordance with an embodiment of the present invention include: stainless steel, rigid plastic resin, polycarbonate, and titanium.

Up to this point, the described embodiments of the ophthalmic shunts have been made of hydrogels. Embodiments of the present invention, however, are not limited to hydrogel ophthalmic shunts. Examples of other materials that can be used to manufacture ophthalmic shunts in accordance with an embodiment of the present invention include: elastomeric materials, such as silicone rubber and polyurethane; glass; ceramic; polycarbonate; acrylic resin; stainless steel; titanium; silver; gold; and platinum.

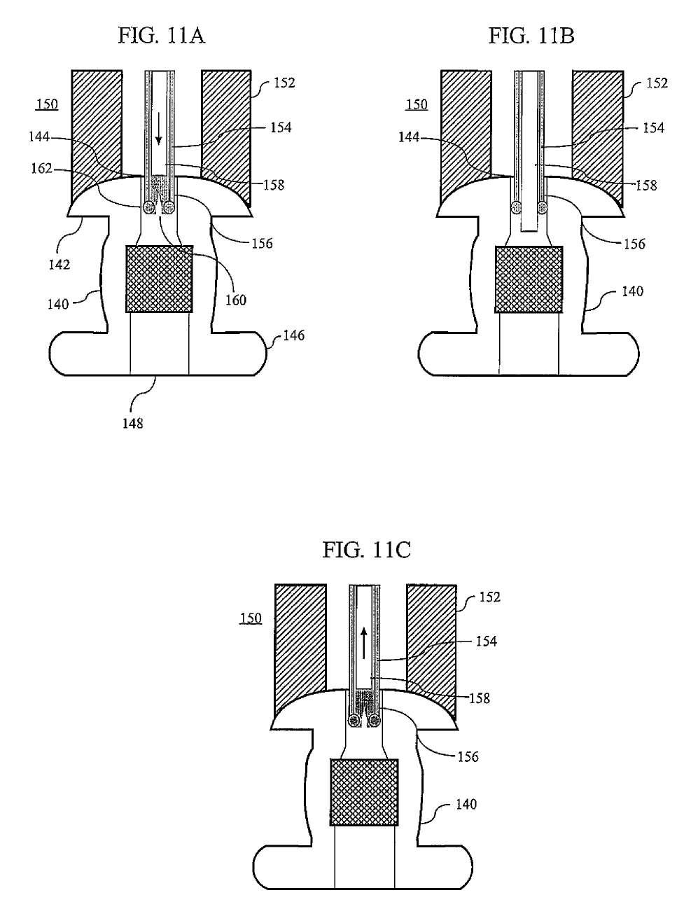

FIGS. 10A-10C illustrate an apparatus and method according to an embodiment of the present invention, in which engagement and release of an ophthalmic shunt are mechanically directed. In other words, hydration of the ophthalmic shunt is not involved in the engagement or release of the shunt with respect to an insertion tool. In contrast to the automatic release of the shunt by hydration by the aqueous humor inside the eye, and the injection of fluid into the shunt via the stabilizer, which still requires a period of time (though likely shorter than the automatic release) for the shunt to hydrate, the embodiments of FIGS. 10A-10C and FIGS. 11A-11C may provide a solution for surgeons that desire a more positive action, or mechanical engagement and release of the shunt with respect to the insertion tool. It will be understood that such mechanical engagement and release mechanisms could be used with hydrogel shunts and elastomeric shunts, as well as shunts made of more rigid materials.

FIG. 10A shows an insertion tool 130, including a stabilizer 132 and an engager 134. The engager 134 includes a hollow tube 136 and a plunger 138 movably disposed within the hollow tube 136. Also, as shown in FIG. 10A, the plunger 138 has a distal end that is larger than an internal diameter of the hollow tube 136.

To use the insertion tool 130, a surgeon contacts the stabilizer 132 to the head 142 of the transcorneal shunt 140, surrounding the head hole 144, and thereby inserting engager 134 (both the hollow tube 136 and the plunger 138) into the head hole 144. Next, to secure the shunt 140 on the insertion tool, the surgeon retracts plunger 136 into hollow tube 136 (shown in FIG. 10A), elastically expanding a distal end of the hollow tube 136 (shown in FIG. 10B) to engage the shunt 140. Then, to release the shunt 140 from the insertion tool 130, after insertion of the shunt 140 into a corneal incision, the hollow tube 136 is held in place, and the surgeon pushes the plunger back down (shown in FIG. 10C), elastically contracting the distal end of the hollow tube 136. Finally, the surgeon pulls the insertion tool 130, removing the contact between the stabilizer 132 and the head 142, and sliding the engager 136 out of the head hole 144.

Thus, FIGS. 10A-10C show an apparatus having the following: a transcorneal shunt 140 with a head 142 and a foot 146, each having a hole therein 144, 148; and an insertion tool 130 to insert the transcorneal shunt 140 into a corneal incision. The insertion tool 130 includes: a stabilizer 132, and an engager 134 protruding from the stabilizer 132 and releasably engaging the shunt 140. The engager 134 includes a hollow tube 136, and a plunger 138 movably disposed within the hollow tube 136. FIGS. 10A-10C also show that when the stabilizer 132 contacts the head 142 and surrounds the corresponding hole (head hole 144), the engager 134 is inserted, at least partially, into the corresponding hole (head hole 144).

Additionally, FIGS. 10A-10C (in conjunction with FIG. 2) also show a method of implanting a transcorneal shunt 140 into a cornea, including the operations: inserting an engager 134 of an insertion tool 130 into one of a head and a foot hole 144,148 of the shunt 140, and contacting a stabilizer 132 of the insertion tool 130, from which the engager 136 protrudes, to the one of the head 142 and foot 146 corresponding to the one of the head and foot holes 144, 148 the engager 134 is inserted into, the engager including a hollow tube 136 and a plunger 138 that is movably disposed within the hollow tube 136; inserting a portion of the shunt 140 through a corneal incision to position and seat the shunt 140; and releasing the shunt 140 from the engager 134, wherein releasing the shunt 140 from the engager includes extending the distal end of the plunger 138 to a position outside of the hollow tube 136.

FIGS. 11A-11C illustrate an apparatus and method according to another embodiment of the present invention, in which engagement and release of an ophthalmic shunt are mechanically directed.

FIG. 11A shows an insertion tool 150, including a stabilizer 152 and an engager 154. The engager includes a hollow tube 156 and a plunger 158 movably disposed within the hollow tube 156. As shown in FIG. 11A, the hollow tube 156 has a slotted tip 160, and a distal end 162 of the hollow tube 156 has a thickness greater than a thickness of a remainder of the hollow tube 156.

To use the insertion tool 50, a surgeon contacts the stabilizer 152 to the head 142 of the transcorneal shunt 140, surrounding the head hole 144, and thereby inserting engager 154 (both the hollow tube 156 and the plunger 158) into the head hole 144. (Initially, the plunger 158 is withdrawn inside of the hollow tube 156). Next, to secure the shunt 140 on the insertion tool, the surgeon pushes plunger 156 down into the distal end 162 of the hollow tube 156 (shown in FIG. 11A), elastically expanding the distal end 162 of the hollow tube 156 (shown in FIG. 11B) to engage the shunt 140. Then, to release the shunt 140 from the insertion tool 150, after insertion of the shunt 140 into a corneal incision, the hollow tube 156 is held in place, and the surgeon retracts the plunger 158 from the distal end 162 of the hollow tube 156 (shown in FIG. 11C), elastically contracting the distal end 162 of the hollow tube 156. Finally, the surgeon pulls the insertion tool 150, removing the contact between the stabilizer 152 and the head 142, and sliding the engager 156 out of the head hole 144.

Thus, FIGS. 11A-11C show an apparatus having the following: a transcorneal shunt 140 with a head 142 and a foot 146, each having a hole therein 144, 148; and an insertion tool 150 to insert the transcorneal shunt 140 into a corneal incision. The insertion tool 150 includes: a stabilizer 152, and an engager 154 protruding from the stabilizer 152 and releasably engaging the shunt 140. The engager 154 includes a hollow tube 156, and a plunger 158 movably disposed within the hollow tube 156. FIGS. 11A-11C also show that when the stabilizer 152 contacts the head 142 and surrounds the corresponding hole (head hole 144), the engager 154 is inserted, at least partially, into the corresponding hole (head hole 144).

Additionally, FIGS. 11A-11C (in conjunction with FIG. 2) also show a method of implanting a transcorneal shunt 140 into a cornea, including the operations: inserting an engager 154 of an insertion tool 150 into one of a head and a foot hole 144,148 of the shunt 140, and contacting a stabilizer 152 of the insertion tool 150, from which the engager 156 protrudes, to the one of the head 142 and foot 146 corresponding to the one of the head and foot holes 144, 148 the engager 154 is inserted into, the engager including a hollow tube 156 and a plunger 158 that is movably disposed within the hollow tube 156; inserting a portion of the shunt 140 through a corneal incision to position and seat the shunt 140; and releasing the shunt 140 from the engager 154, wherein releasing the shunt 140 from the engager 154 includes retracting the plunger 158 from the distal end 162 of the hollow tube 156.

Up to this point, the embodiments have been described in terms of extra-cameral implantation of the transcorneal shunt. By extra-cameral implantation, Applicants mean implantation from outside the anterior chamber of the eye. Embodiments of the present invention, however, are not limited to extra-cameral shunt insertion.

Although extra-cameral transcorneal shunt implantation has been shown to be successful, there can be attendant issues that arise with regard to extra-cameral transcorneal shunt implantation. For example, since the foot of the shunt is larger than the head, a longer corneal incision is needed to pass the foot through the cornea. Depending on the size of the incision, this can be traumatic, and may result in aqueous leakage around the shunt. Additionally, because the surgeon is pushing the shunt through the corneal incision from the outside, the forces applied to the cornea during extra-cameral implantation can tend to flatten the anterior chamber, which can make full insertion difficult and may result in damage to the iris or lens.

Further, viscoelastic is a material that exhibits both viscous and elastic characteristics when undergoing deformation, and is routinely used to manage and/or maintain the shape of the anterior chamber and protect corneal endothelium during ophthalmic procedures. During extra-cameral transcorneal shunt implantation, since the foot hole is open, if viscoelastic is employed during such a procedure, there is potential to clog the filter/flow regulator with viscoelastic.

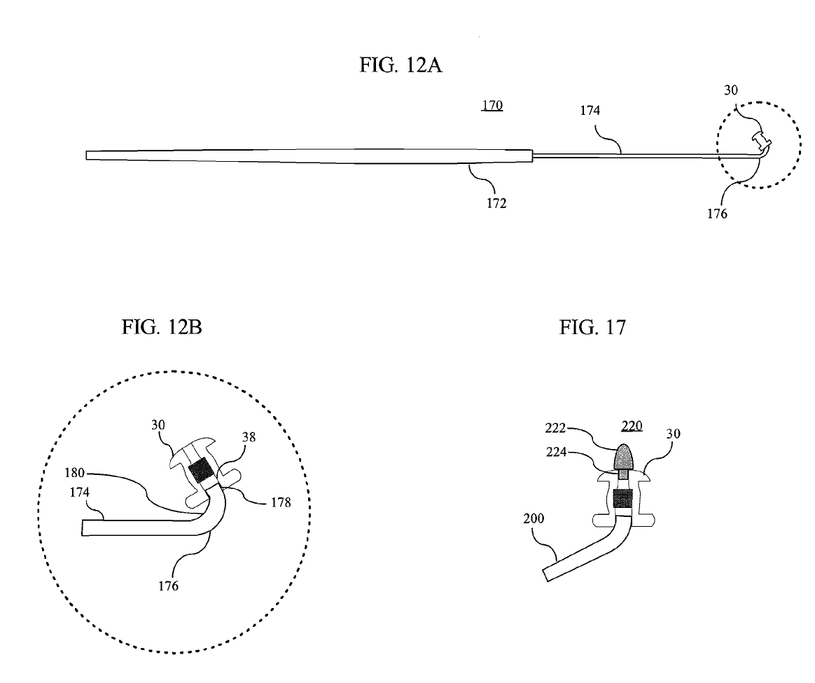

FIG. 12A illustrates an embodiment of the present invention for use in intra-cameral implantation of a transcorneal shunt. And FIG. 12B illustrates a cross sectional detailed view of a circled region of FIG. 12A. By intra-cameral implantation, Applicants mean implantation from within the anterior chamber of the eye. FIGS. 12A and 12B show an insertion tool 170, including a handle 172, a shaft portion 174, a stabilizing portion 176, and an engaging portion 178. Additionally, FIG. 12A shows an ophthalmic shunt, for example, transcorneal shunt 30 engaged with insertion tool 170.

As shown in more detail in FIG. 12B, according to one embodiment, stabilizing portion 176 has an acute bend 180. According to one embodiment, prior to formation of the bend 180, the shaft portion 174, the stabilizing portion 176, and the engaging portion 178 have the same diameter. Thus, after formation of the bend 180, at an interior of the bend 180, for example, the left side of the bend 180 in FIG. 12B, the material of the stabilizing portion is compressed. In contrast, after formation of the bend 180, at an exterior of the bend 180, for example, the right side of the bend 180 in FIG. 12B, the material of the stabilizing portion is stretched. Thus, compression of the material at the interior of the bend 180 not only provides strength and rigidity for the contact between the insertion tool 170 and the shunt 30, but also acts as a stop, and so that the engaging portion 178, which protrudes from the stabilizing portion 176 (stabilizing portion 176 including bend 180), cannot damage or displace the filter/flow restrictor of the shunt 30.

Thus, FIGS. 12A and 12B show an apparatus having the following: a hydrogel transcorneal shunt 30 with a head 32 and a foot 34, each having a hole 36, 38 therein; and an insertion tool 170 to insert the transcorneal shunt 30 into a corneal incision. The insertion tool 170 includes a shaft portion 174, a stabilizing portion 176 extending from the shaft portion 174, and an engaging portion 178 extending from the stabilizing portion 176 and releasably engaging the shunt 30. The engaging portion 178 has at least a portion of an inserted portion thereof that is sized to be greater than a size of the foot hole 38 when the shunt 30 is dehydrated, and less than the size of the foot hole 38 when the shunt 30 is hydrated.

According to one embodiment, though not shown in FIGS. 12A and 12B, at least a portion of the engaging portion inserted into the shunt includes an irregularity to enhance shunt engagement. It will be appreciated that the irregularities depicted in other embodiments could also be employed in the embodiment depicted in FIGS. 12A and 12B, but that a figure depicting such an embodiment is omitted for brevity.

Additionally, according to one embodiment, though not shown in FIGS. 12A and 12B, the shaft portion, the stabilizing portion, and the engaging portion comprise hollow tubing. Further, according to one embodiment, though not shown in FIGS. 12A and 12B, the shaft portion includes a luer connection to accommodate a syringe to inject fluid into the tubing. It will be appreciated that the hollow tubing and luer connection depicted in other embodiments could also be employed in the embodiment depicted in FIGS. 12A and 12B, but that figures depicting such embodiments are omitted for brevity.

FIGS. 13-15 illustrate an apparatus and method according to an embodiment of the present invention.

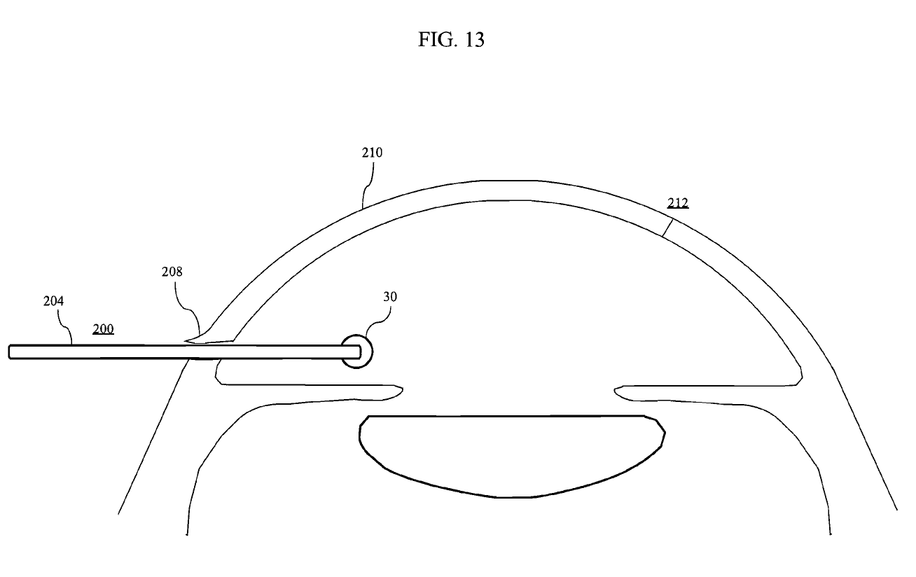

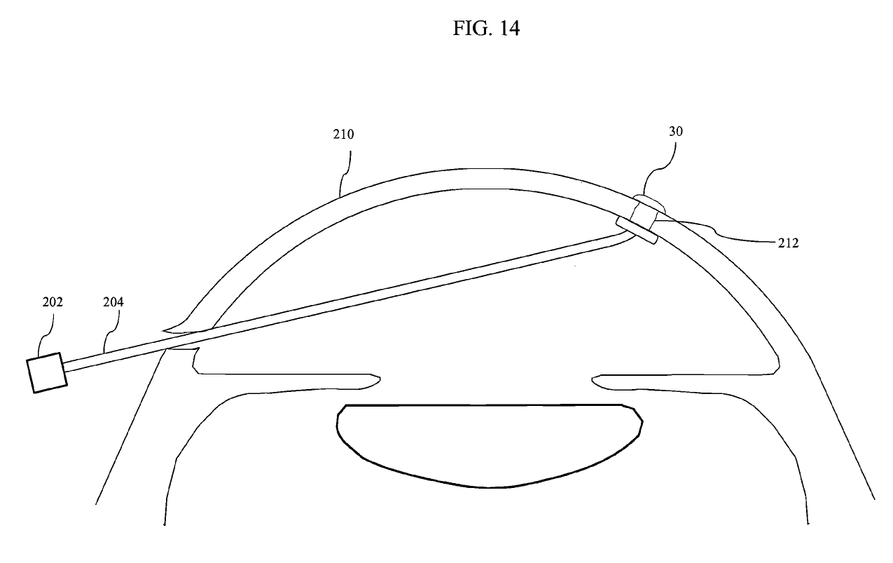

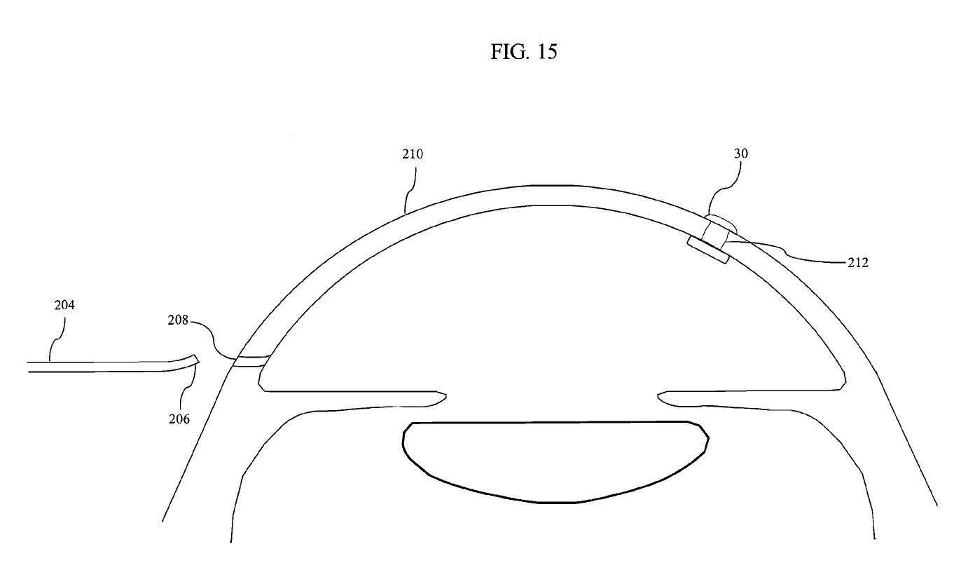

For intra-cameral implantation of a transcorneal shunt, the shunt, for example, hydrogel transcorneal shunt 30, is engaged with an insertion tool. As shown in FIG. 13, hydrogel transcorneal shunt 30 is already engaged with insertion tool 200, which includes engager 206 (see FIG. 15). According to one embodiment, engaging the hydrogel transcorneal shunt 30 with the insertion tool includes hydrating the shunt 30, inserting engager 206 into foot hole 38 of the shunt 30, and dehydrating the shunt 30. As shown in FIGS. 13-15, insertion tool 200 includes handle 202 (see FIG. 14), a stabilizer 204, and engager 206 protruding from the stabilizer 204 (see FIG. 15). In FIG. 13, the view of shunt 30 is an axial view, looking at the foot of shunt 30.

After the shunt 30 is engaged with the insertion tool, the surgeon makes a paracentesis incision, or entry incision 208 in cornea 210. The surgeon also makes an incision at the implantation site, or an implant incision 212 in the cornea 210. According to one embodiment, making the entry incision 208 in the cornea 210 includes making an incision approximately parallel (as shown in FIG. 13) to a corresponding iris near where the cornea meets a corresponding limbus. Additionally, according to one embodiment, making the implant incision 212 in the cornea 210 includes making an incision approximately perpendicular to the cornea. These respective orientations of these two incisions promote wound healing, and in the case of the implant incision, also correctly orients the shunt.

As shown in FIG. 13, the surgeon inserts the insertion tool-engaged shunt 30 through the entry incision 208, and maneuvers the shunt 30 to the implant incision 212. The surgeon then inserts the shunt, head-first, into the cornea 210 through the implant incision 212 (FIG. 14). After the shunt 30 is properly positioned and seated in the cornea 210, the shunt 30 is released from the insertion tool 200. According to one embodiment, releasing the shunt 30 from the insertion tool 200 includes re-hydrating the shunt 30. According to one embodiment, such re-hydration includes hydrating the shunt 30 with aqueous humor from the cornea's 210 anterior chamber to automatically release the shunt 30 from the insertion tool 200. According to one embodiment, such re-hydration includes admitting fluid to the shunt 30 via the stabilizing portion 204.

Finally, as shown in FIG. 15, the insertion tool 200 is removed from the insertion incision 208. Thus, as illustrated in FIGS. 13-15, the route of implantation is through the anterior chamber of the eye.

Thus, FIGS, 13-15 show a method of implanting a transcorneal shunt 30 into a cornea 210, the shunt 30 having a head 32 and a foot 34, each having a hole therein 36, 38. The method includes engaging an insertion tool 200 with the foot hole 38 of the shunt 38, and making an entry incision 208 in the cornea 210. Additionally, the method includes: inserting the shunt 30, while still engaged with the insertion tool 200, through the entry incision 208; and making an implant incision 212 in the cornea 210. Further, the method includes inserting the head 32 of the shunt 30 through the implant incision 212 to position and seat the shunt 30, and releasing the shunt 30 from the insertion tool 200.

Additionally, FIGS, 13-15 show an apparatus having the following: a hydrogel transcorneal shunt 30 with a head 32 and a foot 34, each having a hole therein 36, 38; and an insertion tool 200 to insert the transcorneal shunt 30 into a corneal incision 212. The insertion tool 200 includes a stabilizer 204, and an engager 206 protruding from the stabilizer 204 and releasably engaging the shunt 30, the engager 206 automatically releasing the shunt 30 subsequent the shunt's 30 insertion into the corneal incision 212.

According to one embodiment, the stabilizer 204 and engager 206 are each approximately 0.5 mm in diameter, and together, are about 20 mm long.

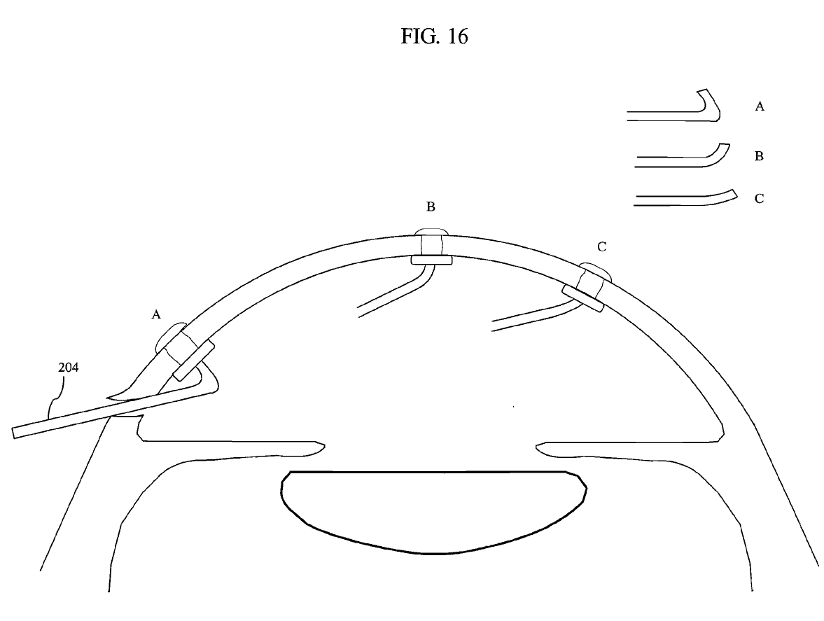

FIG. 16 illustrates variations of insertion tools according to embodiments of the present invention. FIG. 16 shows how the angle of bend in the insertion tool can be selected to accommodate a desired location of the implant incision relative to the entry incision. In example A, the angle is acute to accommodate a positioning of the implant incision close to the entry incision. In example C, the angle is obtuse to accommodate the implant incision being trans-corneally positioned with respect to the entry incision. And in example B, the angle is in between the angles of examples A and C, accommodating a median positioning of the implant incision with respect to the entry incision.

FIG. 17 illustrates a cross-sectional view of an incision-making device according to an embodiment of the present invention. FIG. 17 shows insertion tool 200 engaged with transcorneal shunt 30, and thus, engager 206 is inserted into foot hole 38 of shunt 30. An incision-making device 220 includes a cutting edge 222 and a support boss 224. Support boss 224 is inserted into head hole 36 to secure the incision-making device 220 on the shunt 30. In use, according to one embodiment, the surgeon inserts the incision-making device 220, which is secured to shunt 30, which in turn is installed on insertion tool 200, through an entry incision and guides the device 220 to an implantation site. The surgeon then employs cutting edge 222 to make the implant incision into which the shunt 30 is inserted.

According to one embodiment, the insertion tool and shunt are supplied together in sterile packaging, with the shunt already engaged with the insertion tool.

Inserting the shunt via the intra-cameral route involves passing the shunt head through the cornea instead of the foot. In experiments, it has been determined that because the head is smaller in diameter than the foot, and because it is dome-shaped, a smaller corneal implant incision can be used for an intra-cameral transcorneal shunt implantation. In experiments, shunts were successfully implanted through 1.48 mm incisions, which are considerably smaller than those used in a typical extra-cameral transcorneal shunt implantation.

Thus, because the head is smaller than the foot, passing the shunt through the cornea head-first requires a shorter incision. Also, because the shunt is inserted from within the anterior chamber, the forces applied to the cornea during insertion of the shunt tend to deepen the chamber instead of flattening it. Further, because the insertion tool is inserted into the shunt's foot hole, viscoelastic can be used to aid implantation, as long as it is removed before the shunt is released.

Although a few embodiments of the present invention have been shown and described, the present invention is not limited to the described embodiments. Instead, it will be appreciated by those skilled in the art that changes may be made to these embodiments without departing from the principles and spirit of the invention, the scope of which is defined by the claims and their equivalents.

What is claimed is:

1. An apparatus, comprising:
a transcorneal shunt with a head and a foot, each having a hole therein; and
an insertion tool to insert the transcorneal shunt into a corneal incision, the insertion tool comprising a stabilizer, and an engager protruding from the stabilizer and releasably engaging the shunt,
wherein when the stabilizer contacts one of the head and foot and surrounds the corresponding hole, the engager is inserted, at least partially, into the corresponding hole, and at least a portion of an inserted length of the engager comprises an irregularity to enhance shunt engagement.

2. The apparatus according to claim 1, wherein the stabilizer comprises hollow tubing.

3. The apparatus according to claim 2, wherein the stabilizer comprises hypodermic tubing.

4. The apparatus according to claim 2, further comprising a luer connection to accommodate a syringe to inject fluid into the tubing.

5. The apparatus according to claim 1, wherein a first end of the stabilizer, at which the engager protrudes, has an angle formed therein.

6. The apparatus according to claim 5, wherein:
the stabilizer contacts the foot;
the shunt is inserted into the corneal incision intra-camerally via an entry incision; and
the angle is acute to accommodate a positioning of the corneal incision close to the entry incision.

7. The apparatus according to claim 1, wherein:
the stabilizer contacts the foot;
the shunt is inserted into the corneal incision intra-camerally via an entry incision; and
the angle is obtuse to accommodate the corneal incision being trans-corneally positioned with respect to the entry incision.

8. The apparatus according to claim 1, wherein a first end of the stabilizer, at which the engager protrudes, has a curve formed therein.

9. The apparatus according to claim 1, wherein a distal end of the stabilizer, at which the engager protrudes, is recessed to accommodate a shape of the head.

10. The apparatus according to claim 9, wherein the distal end of the stabilizer is machined to a radius to conform to the shape of the head.

11. The apparatus according to claim 1, wherein a distal end of the stabilizer, at which the engager protrudes, is approximately perpendicular to a side of the stabilizer immediately adjacent to the distal end of the stabilizer.

12. The apparatus according to claim 1, wherein the stabilizer and the engager are integrally formed as a unitary construction.

13. The apparatus according to claim 1, wherein the at least portion of the inserted length of the engager comprises a plurality of irregularities to enhance shunt engagement.

14. The apparatus according to claim 1, wherein the irregularity comprises a raised feature to engage the shunt.

15. The apparatus according to claim 1, wherein the irregularity comprises a tapered undercut.

16. The apparatus according to claim 1, wherein the irregularity comprises a groove.

17. The apparatus according to claim 16, wherein the groove comprises one of a v-groove, a rounded groove, and a square groove.

18. The apparatus according to claim 1, wherein the irregularity comprises a helical thread.

19. The apparatus according to claim 1, wherein the irregularity comprises a ball-shape.

20. The apparatus according to claim 1, wherein the shunt comprises one of silicone rubber and polyurethane.

21. The apparatus according to claim 1, wherein the shunt comprises at least one of glass, ceramic, polycarbonate, acrylic resin, stainless steel, titanium, silver, gold, and platinum.

22. The apparatus according to claim 1, wherein the stabilizer comprises one of stainless steel, rigid plastic resin, polycarbonate, and titanium.

23. The apparatus according to claim 1, wherein the engager comprises one of stainless steel, rigid plastic resin, polycarbonate, and titanium.

24. The apparatus according to claim 1, wherein the insertion tool further comprises a handle connected to the stabilizer to aid manipulation of the insertion tool.

25. The apparatus according to claim 1, further comprising:
an incision-making device,
wherein the engager is inserted into the foot hole, and a portion of the incision-making device is inserted into the head hole, so that during an intra-cameral insertion of the transcorneal shunt, the incision making device is used to make the corneal incision into which the shunt is inserted.

26. An apparatus, comprising:
a hydrogel transcorneal shunt with a head and a foot, each having a hole therein; and
an insertion tool to insert the transcorneal shunt into a corneal incision, the insertion tool comprising a stabilizer, and an engager protruding from the stabilizer and releasably engaging the shunt,
wherein when the stabilizer contacts one of the head and foot and surrounds the corresponding hole, the engager is inserted, at least partially, into the corresponding hole, and the engager has at least a portion of an inserted portion thereof that is sized to be greater than a size, when the shunt is dehydrated, of the one of the head and foot holes the engager is inserted into, and less than the size, when the shunt is hydrated, of the one of the head and foot holes the engager is inserted into.

27. The apparatus according to claim 26, wherein a diameter of the portion of the inserted portion of the engager is greater than the diameter, when the shunt is dehydrated, of the of the one of the head and foot holes the engager is inserted into, and less than the diameter, when the shunt is hydrated, of the one of the head and foot holes the engager is inserted into.

28. An apparatus, comprising:
a transcorneal shunt with a head and a foot, each having a hole therein; and
an insertion tool to insert the transcorneal shunt into a corneal incision, the insertion tool comprising a stabilizer, and an engager protruding from the stabilizer and releasably engaging the shunt, the engager comprising a hollow tube, and a plunger movably disposed within the hollow tube,
wherein when the stabilizer contacts one of the head and foot and surrounds the corresponding hole, the engager is inserted, at least partially, into the corresponding hole.

29. The apparatus according to claim 28, wherein:
the plunger has a distal end larger than an internal diameter of the hollow tube; and
the distal end of the plunger is retracted into the hollow tube to elastically expand a distal end of the hollow tube to engage the shunt.

30. The apparatus according to claim 28, wherein:
the hollow tube has a slotted tip;
a distal end of the hollow tube has a thickness greater than a thickness of a remainder of the hollow tube; and
the plunger is pushed down into the distal end of the hollow tube to elastically expand the distal end of the hollow tube to engage the shunt.

31. An apparatus, comprising:
a hydrogel transcorneal shunt with a head and a foot, each having a hole therein; and
an insertion tool to insert the transcorneal shunt into a corneal incision, the insertion tool comprising a handle to aid in manipulating the insertion tool, a stabilizer, comprising hypodermic tubing extending from the handle, and an engager protruding from the stabilizer and releasably engaging the shunt, the engager having a diameter greater than a diameter, when the shunt is dehydrated, of the of the one of the head and foot holes the engager is inserted into, and less than the diameter, when the shunt is hydrated, of the one of the head and foot holes the engager is inserted into.

32. An apparatus, comprising:
a hydrogel transcorneal shunt with a head and a foot, each having a hole therein; and
an insertion tool to insert the transcorneal shunt into a corneal incision, the insertion tool comprising a stabilizer, and an engager protruding from the stabilizer and releasably engaging the shunt, the engager automatically releasing the shunt subsequent the shunt's insertion into the corneal incision.

33. A method of implanting a hydrogel transcorneal shunt into a cornea, comprising:
hydrating the shunt, the shunt having a head and a foot, each having a hole therein;
inserting an engager of an insertion tool into one of the head and foot holes of the hydrated shunt;
dehydrating the shunt subsequent to insertion of the engager;
inserting the dehydrated shunt into a corneal incision; and
re-hydrating the shunt to release the shunt from the insertion tool.

34. The method according to claim 33, wherein re-hydrating the shunt to release the shunt from the insertion tool comprises hydrating the shunt with aqueous humor from the cornea's anterior chamber to automatically release the shunt from the insertion tool.

35. The method according to claim 33, wherein re-hydrating the shunt to release the shunt from the insertion tool comprises admitting fluid to the shunt via the stabilizer.

36. The method according to claim 33, wherein the engager is inserted into the head hole and the shunt is inserted into the corneal incision from outside the eye.

37. The method according to claim 33, wherein the engager is inserted into the foot hole and the shunt is inserted into the corneal incision intra-camerally.

38. A method of implanting a transcorneal shunt into a cornea, comprising:
inserting an engager of an insertion tool into one of a head and a foot hole of the shunt, and contacting a stabilizer of the insertion tool, from which the engager protrudes, to the one of the head and foot corresponding to the one of the head and foot holes the engager is inserted into, the engager comprising a hollow tube and a plunger that is movably disposed within the hollow tube;
inserting a portion of the shunt through a corneal incision to position and seat the shunt; and
releasing the shunt from the engager,
wherein releasing the shunt from the engager comprises one of extending the distal end of the plunger to a position outside of the hollow tube and retracting the plunger from the distal end of the hollow tube.

39. The method according to claim 38, further comprising:
engaging the shunt with the engager,
wherein the plunger has a distal end larger than an internal diameter of the hollow tube, engaging the shunt with the engager comprises retracting the distal end of the shunt into the hollow tube to elastically expand a distal end of the hollow tube to engage the shunt, and releasing the shunt from the engager comprises extending the distal end of the plunger to a position outside of the hollow tube.

40. The method according to claim 38, further comprising:
engaging the shunt with the engager,
wherein the hollow tube has a slotted tip, a distal end of the hollow tube is thicker than a remainder of the hollow tube, engaging the shunt with the engager comprises pushing the plunger into the distal end of the hollow tube to elastically expand a distal end of the hollow tube to engage the shunt, and releasing the shunt from the engager comprises retracting the plunger from the distal end of the hollow tube.

41. The method according to claim 38, wherein the engager is inserted into the head hole and the shunt is inserted into the corneal incision from outside the eye.

42. The method according to claim 38, wherein the engager is inserted into the foot hole and the shunt is inserted into the corneal incision intra-camerally.

43. A method of implanting a transcorneal shunt into a cornea, the shunt having a head and a foot, each having a hole therein, the method comprising:
engaging an insertion tool with a foot hole of the shunt;
making an entry incision in the cornea;
inserting the shunt, while still engaged with the insertion tool, through the entry incision;
making an implant incision in the cornea;
inserting the head of the shunt through the implant incision to position and seat the shunt; and
releasing the shunt from the insertion tool.

44. The method according to claim 43, wherein the engaging the insertion tool with the foot hole of the shunt comprises inserting an engager of the insertion tool into the foot hole of the shunt, and contacting a stabilizer of the insertion tool, from which the engager protrudes, to the foot.

45. The method according to claim 43, wherein:
the shunt comprises a hydrogel shunt; and
the engaging the insertion tool with the foot hole of the shunt comprises hydrating the shunt, inserting an engager of an insertion tool into a foot hole of the shunt, the engager protruding from a stabilizer of the insertion tool, and dehydrating the shunt.

46. The method according to claim 45, wherein the releasing the shunt from the insertion tool comprises re-hydrating the shunt.

47. The method according to claim 46, wherein re-hydrating the shunt comprises hydrating the shunt with aqueous humor from the cornea's anterior chamber to automatically release the shunt from the insertion tool.

48. The method according to claim 46, wherein re-hydrating the shunt comprises admitting fluid to the shunt via the stabilizer.

49. The method according to claim 43, wherein the making an entry incision in the cornea comprises making an incision approximately parallel to a corresponding iris near where the cornea meets a corresponding limbus.

50. The method according to claim 43, wherein the making an implant incision in the cornea comprises making an incision approximately perpendicular to the cornea.

51. An apparatus, comprising:
a hydrogel transcorneal shunt with a head and a foot, each having a hole therein; and
an insertion tool to insert the transcorneal shunt into a corneal incision, the insertion tool comprising a shaft portion, a stabilizing portion extending from the shaft portion, and an engaging portion extending from the stabilizing portion and releasably engaging the shunt,
wherein the engaging portion has at least a portion of an inserted portion thereof that is sized to be greater than a size of the foot hole when the shunt is dehydrated, and less than the size of the foot hole when the shunt is hydrated.

52. The apparatus according to claim 51, wherein the stabilizing portion comprises an acute bend in the insertion tool.

53. The apparatus according to claim 52, wherein prior to formation of the bend, the shaft portion, the stabilizing portion, and the engaging portion have substantially the same diameter.

54. The apparatus according to claim 51, wherein the at least portion of the inserted portion of the engaging portion comprises an irregularity to enhance shunt engagement.

55. The apparatus according to claim 51, wherein the shaft portion, the stabilizing portion, and the engaging portion comprise hollow tubing.

56. The apparatus according to claim 55, further comprising a luer connection to accommodate a syringe to inject fluid into the tubing.
